SEQUENCE LISTING (8 sequences)

SEQ ID NO: 1 - DNA, 22 bp, Artificial sequence
  PCR primer
gccatcgactacattcgctttc

SEQ ID NO: 2 - DNA, 21 bp, Artificial sequence
  PCR primer
cctccatgagcacgtctgtgt

SEQ ID NO: 3 - DNA, 25 bp, Artificial sequence
  PCR primer
ggagtctatgtccactcaagcattt

SEQ ID NO: 4 - DNA, 21 bp, Artificial sequence
  PCR primer
ttccaagctaccatgccaatc

SEQ ID NO: 5 - DNA, 19 bp, Artificial sequence
  PCR primer
cagccccctggaaagtgat

SEQ ID NO: 6 - DNA, 24 bp, Artificial sequence
  PCR primer
cgtctccaacttatctcctccatt

SEQ ID NO: 7 - DNA, 21 bp, Artificial sequence
  PCR primer
catgtacgttgctatccaggc

SEQ ID NO: 8 - DNA, 21 bp, Artificial sequence
  PCR primer
ctccttaatgtcacgcacgat

(19) United States
(12) Patent Application Publication
(10) Pub. No.: US 2018/0296627 A1
(43) Pub. Date: Oct. 18, 2018

(54) METHOD FOR MODULATING THE EXPRESSION OF THE SREBP-1C, ACC, AND SCD-1 PROTEINS USING LONGAN PERICARP EXTRACT

(71) Applicant: TCI CO., LTD, TAIPEI (TW)

(72) Inventors: Yung-Hsiang Lin, Taipei (TW); I-Hui Chen, Taipei (TW); Kai-Wen Kan, Taipei (TW)

(21) Appl. No.: 15/947,914

(22) Filed: Apr. 9, 2018

(30) Foreign Application Priority Data
Apr. 10, 2017 (TW) 106111885

(51) Int. Cl. A61K 36/77; A61P 1/16

(52) U.S. Cl. CPC A61K 36/77; A61K 2236/333; A61K 2236/331; A61P 1/16

(57) ABSTRACT

The present invention provides a method for reducing the expressions of the sterol regulatory element-binding protein 1C (SREBP-1C), acetyl-CoA carboxylase (ACC), and stearoyl-CoA desaturase 1 (SCD-1), comprising administering to a subject a composition comprising an effective amount of a longan pericarp extract. The longan pericarp extract significantly inhibits lipogenesis, reduces the glutamic-pyruvic transaminase (GPT) index, and protects the liver.

### CROSS-REFERENCE TO RELATED APPLICATION

This application claims priority of Taiwan patent application No. 106111885, filed on Apr. 10, 2017, the content of which is incorporated herein in its entirety by reference.

### BACKGROUND OF THE INVENTION

### 1. Field of the Invention

The present invention relates to a method for modulating the expression of SREBP-1c and ACC and SCD-1 proteins, and more particularly to a method for modulating such proteins expression by administering a longan pericarp extract.

### 2. The Prior Art

Domestic people suffer from liver diseases due to viral infection, drug abuse, long-term alcohol abuse and other reasons. According to the 2015-year statistics of the Department of Health, Executive Yuan Taiwan, liver diseases such as chronic liver disease and cirrhosis are among the ten leading causes of death among the domestic people.

In recent years, the consumption of alcoholic beverages in Taiwan has increased significantly, and the prevalence of alcoholic liver disease has also increased relatively. After long-term ingestion of alcohol, the biochemical and pathological damage caused in human bodies are very complicated, and the most affected tissue is the liver, the main tissue of alcohol metabolism. Liver damage caused by long-term ingestion of alcohol includes: fatty liver, alcoholic hepatitis, and cirrhosis, etc., and Liver cancer may be caused if it is not treated promptly.

Fatty liver is the earliest sign of alcoholic liver disease. Studies have shown that patients with alcoholic fatty liver disease have a greater chance of developing hepatic fibrosis and liver cirrhosis. Another study pointed out that fatty liver has a correlation with the occurrence of liver cancer. Therefore, if we can prevent and treat the fatty liver, we could reduce the occurrence of liver fibrosis and cirrhosis. However, there is currently no effective treatment for fatty liver, and most of the patients in clinical practice are advised to eat less fat foods, consume more fruits and vegetables, exercise properly and keep normal routines, and the most important thing is to prohibit alcohol. Nonetheless, for alcohol dependence patients who suffer from fatty liver, it is very difficult to abstain from drinking for a short period of time. Thus, if it is possible to reduce alcohol intake programmatically and eat foods that can reduce fatty liver simultaneously, the maximum therapeutic effect can be achieved. Because the liver is the vital organs which are responsible for the detoxification or metabolism in the human bodies, and alcohol and hepatitis virus infections are the two major causes of liver diseases, the search for antioxidant foods that prevent or reduce liver damage is an important issue in nutrition medicine.

Longan is rich in July-September, and is mainly produced in Tainan, Taiwan, and the annual output in Taiwan is between 100,000 and 130 thousand tons. After baking and shelling, it is dried longan, which can be used as food supplements and can be stored for a long time. However, a large amount of longan pericarp by-products are produced during processing. Therefore, it would be a great blessing for human beings to be able to use these by-products that can only be discarded for further effective use and have a health-care effect on liver diseases.

### SUMMARY OF THE INVENTION

To solve the foregoing problem, one objective of the present invention is to provide a method for reducing the expressions of the sterol regulatory element-binding protein 1C (SREBP-1C), acetyl-CoA carboxylase (ACC), and stearoyl-CoA desaturase 1 (SCD-1), comprising administering to a subject a composition comprising an effective amount of a longan pericarp extract, wherein the longan pericarp extract is obtained by solvent extraction of a longan pericarp, and the solvent is water, alcohol, or a mixture of water and alcohol, and the composition further comprises a pharmaceutically acceptable carrier.

In one embodiment of the present invention, the composition is a powder, granule, liquid, gel or paste.

In one embodiment of the present invention, the extraction is performed from 0.5 to 3 hours, the extraction is performed at a temperature from 50 to 100° C., a liquid-to-solid ratio of the solvent to the longan pericarp is from 20:1 to 1:1, and the composition is at a concentration of at least 4 mg/mL.

The method disclosed herein can effectively inhibit the formation of fatty liver, prevent liver damage, lower the glutamic-pyruvic transaminase (GPT) value, and reduce the hepatic lipogenesis synthesis related genes to achieve the liver protection effect.

The following drawings form part of the present specification and are included here to further demonstrate some aspects of the present invention, which can be better understood by reference to one or more of these drawings, in combination with the detailed description of the embodiments presented herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the relative fat formation levels in the HepG2 cells treated with or without the 4 mg/mL of the longan pericarp extract.

FIG. 2 is a micrograph showing the HepG2 cells treated with or without 4 mg/mL of the longan pericarp extract and stained with oil red O.

FIG. 3 shows the relative GPT index levels in the HepG2 cells pre-treated with or without the 4 mg/mL of the longan pericarp extract before treated with H₂O₂.

FIG. 4 shows a fluorescent staining image of the HepG2 cells pre-treated with or without 4 mg/mL of the longan pericarp extract before treated with H₂O₂.

FIG. 5 shows the relative gene expression levels of SREBP-1c, ACC and SCD-1 in the HepG2 cells pre-treated with 2 mg/mL or 4 mg/mL of the longan pericarp extract.

### DETAILED DESCRIPTION OF THE PREFERRED EMBODIMENT

The present invention provides a method for reducing the expressions of SREBP-1C, ACC, and SCD-1, comprising administering to a subject a composition comprising an effective amount of a longan pericarp extract. The longan pericarp extract of the present invention is obtained by solvent extraction at different temperatures and centrifugation of a longan pericarp. The longan pericarp extract is used to inhibit the formation of fatty liver, prevent liver damage, lower the GTP value, and reduce the hepatic lipogenesis synthesis related genes.

The longan pericarp extract of the present invention is obtained by an extraction method comprising the following steps: (a) the solvent extraction of a longan pericarp is under a liquid-to-solid ratio from 20:1 to 1:1 and is performed from 0.5 to 3 hours at a temperature from 50 to 100° C.; (b) the obtained product is centrifuged; (c) the supernatant is filtrated after centrifugation to obtain a filtrate; (d) the filtrate is concentrated under reduced pressure at a temperature from 45 to 70° C. to obtain a concentrated product; and (e) the concentrated product is subjected to spray drying, and wherein the extraction solvent is water, alcohols, aqueous alcohols or combinations thereof.

The present invention also provides a composition for modulating the expression of SREBP-1c, ACC and SCD-1, comprising an effective amount of longan pericarp extract and a pharmaceutically acceptable carrier, and the composition is a powder, granule, liquid, gel or paste, and the dosage form is provided as food, drink, medicine, reagent or nutritional supplement.

Hereinafter, the detailed extraction method of the longan pericarp extract of the present invention and the test of the longan pericarp extract for inhibiting the formation of fatty liver, preventing liver damage, lowering the GTP index, and lowering the liver fat synthesis related gene test will be described in detail to confirm the effect of the longan pericarp extract.

Preparation of Longan Pericarp Extract
1. Cleaning the longan pericarp to remove debris, and dry it out for subsequent extraction.
2. Crushing the longan pericarp into pieces of about 0.2-0.5 cm.
3. Taking the crushed longan pericarp at a liquid-to-solid ratio from 20:1 to 1:1 at 50 to 100° C. for 0.5 to 3 hours.
4. Cooling the crude extract to room temperature.
5. Centrifuging it at 5000 rpm for 10 minutes for slag removal.
6. Filtering through a 400 mesh filter to remove the filter residue.
7. Concentrating the filtrate under reduced pressure at 45-70° C. to get the longan pericarp extract.

The Fat Formation Test of Longan Pericarp Extract in Liver Cells

Materials:
1. Cell line: HepG2 (ATCC, HB-8065)
2. Culture medium: Dulbecco's Modified Eagle Medium (Gibco, Cat. 12100-038) with 10% fetal bovine serum (Gibco, Cat. 10438-026) and 1% penicillin/streptomycin (Gibco, Cat. 15140-122)
3. Free fatty acids: Oleic acid (Sigma, #O1008)
4. Bovine serum albumin (Bio Basic Inc., Cat. AD0023)
5. Oil red O (Sigma, Cat. O0625)
6. Isopropanol (Echo chemical, PH-3101)
7. 10% Formaldehyde (Echo chemical, Cat. TG1794-4-0000-72NI)
8. Phosphate buffered saline (Gibco, Cat. 14200-075)

Experiment Steps:
1. Seeding 5×10⁵ HepG2 cells per well in 6-well plate and incubating the HepG2 cells at 37° C. overnight.
2. Treated the HepG2 cells with 4 mg/mL longan pericarp extract in the Dulbecco's Modified Eagle medium containing 2% FBS and 1% penicillin/streptomycin for 24 hours.
3. Preparing the medium containing OA-BSA conjugate, 2% FBS, and 1% penicillin/streptomycin.
4. Removing the original medium and replacing with the medium containing OA-BSA conjugate and 4 mg/mL of the longan pericarp extract for 24 hours.
5. Removing the medium and washing the HepG2 cells with 1×PBS for twice.
6. Fixed the HepG2 cells by 10% formaldehyde for 30 min
7. Washing the HepG2 cells with 1×PBS for twice and rinsing by 50% isopropanol for 15 second.
8. Stained the HepG2 cells by oil red O in 60% isopropanol for 1 hour.
9. Observed the HepG2 cells via microscope and dissolving the stain with 100% isopropanol for quantification.
10. Statistics are student t-test using Excel software.

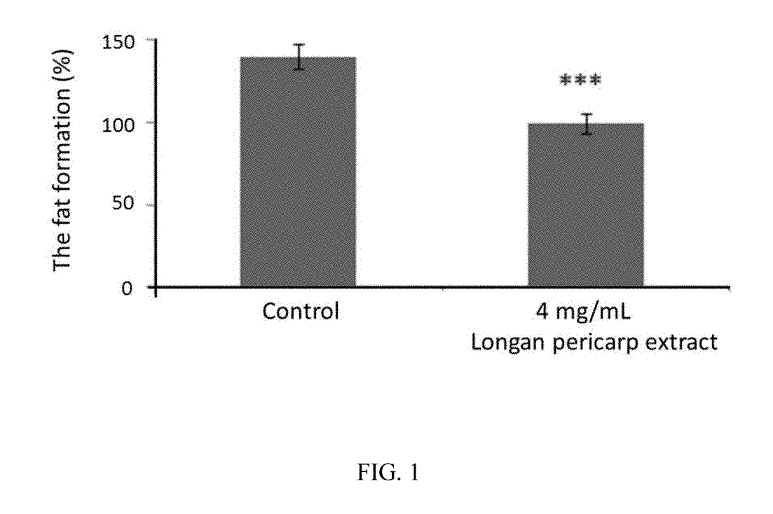

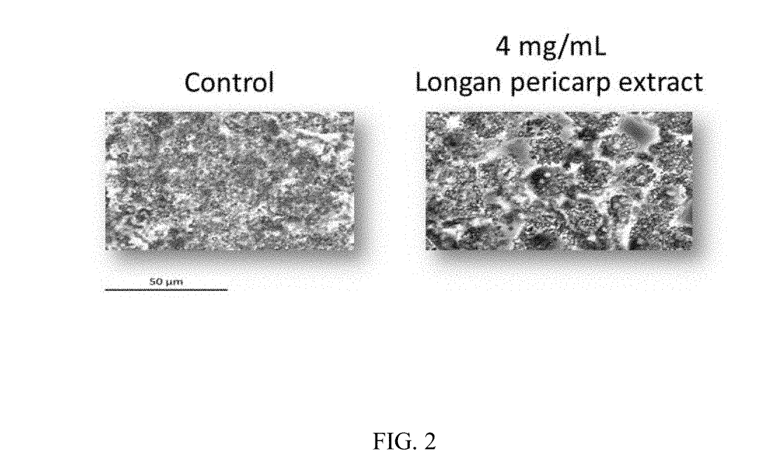

As shown in FIG. 1, the longan pericarp extract inhibits 40.5% of the fat formation in the HepG2 cells treated with 4 mg/mL. The results represent that the longan pericarp extract can effectively reduce the adipogenesis in HepG2 cells, and confirm that the longan pericarp extract can effectively inhibit the formation of fat in HepG2 cells. Besides, as shown in the dyeing micrograph of FIG. 2, the longan pericarp extract inhibits fat formation to achieve the effect of preventing fatty liver.

The GPT (ALT) Test of Longan Pericarp Extract in Injured Liver Cells

Materials:
1. Cell line: HepG2 (ATCC, HB-8065)
2. Culture medium: Dulbecco's Modified Eagle Medium (Gibco, Cat. 12100-038) with 10% fetal bovine serum (Gibco, Cat. 10438-026) and 1% penicillin/streptomycin (Gibco, Cat. 15140-122)
3. ELISA Kit for Alanine Aminotransferase (ALT) (USCN., Cat. SEA207Hu)
4. Phosphate buffered saline (Gibco, Cat. 14200-075)
5. ELISA reader (BioTek)

Experiment Steps:
1. Seeding 2×10⁴ HepG2 cells per well in 24-well plate and incubating the HepG2 cells at 37° C. overnight.
2. Pretreating the HepG2 cells with the longan pericarp extract in the culture medium for 24 hours.
3. Adding H₂O₂ in the final concentration of 500 μM in the HepG2 cells for 6 hours.
4. Collecting 100 μL the supernatant of the culture medium and using the ELISA Kit to determine the level of the Alanine Aminotransferase (ALT or GPT).
5. The statistical significance is performed by student-t-test in Excel software (*: P<0.05; **: P<0.01).

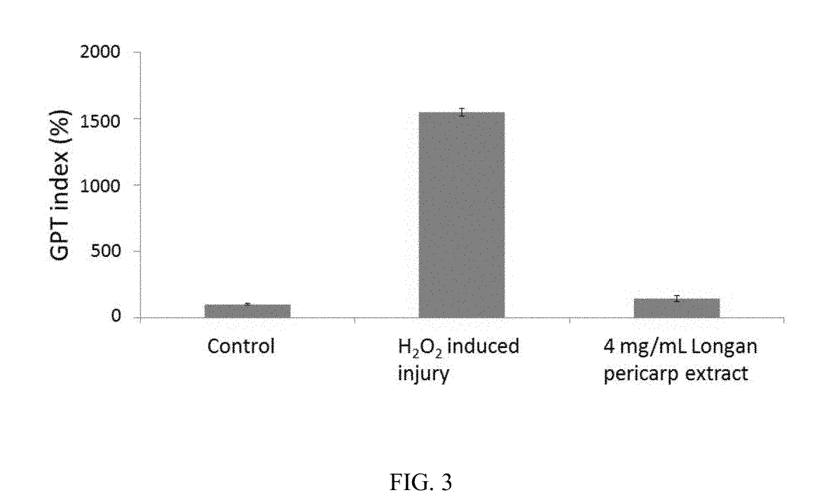

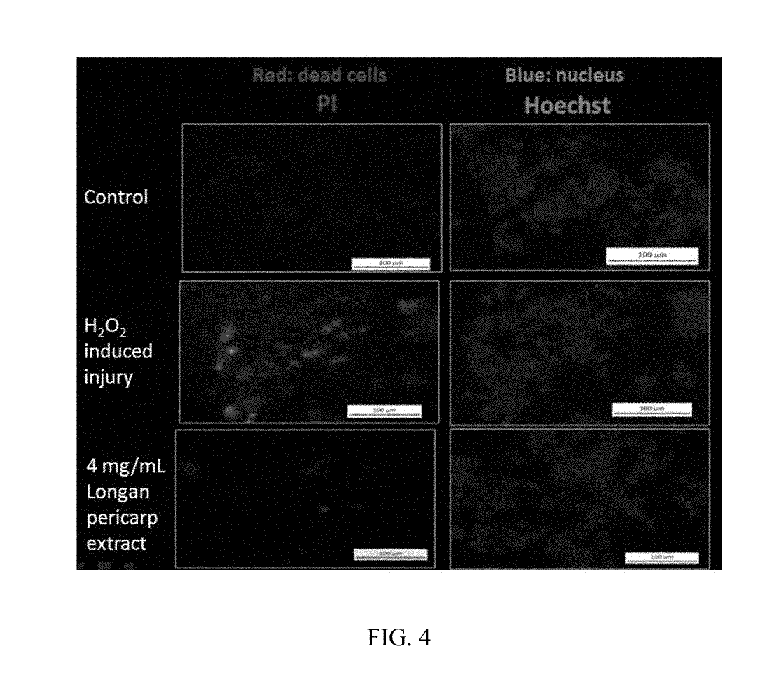

As shown in FIG. 3, after the H₂O₂ injury in HepG2 cells, the GPT index soars. However, the GPT index effectively reduces by 90% in the HepG2 cells pretreated with 4 mg/ml longan pericarp extract before treated with H₂O₂. As shown in FIG. 4, the HepG2 cells undergo a large number of apoptosis after the H₂O₂ injury, and pretreated with longan pericarp extract can protect liver from external stimuli.

The Gene Level Tests of Longan Pericarp Extract in Liver Cells

Materials:
1. Cell line: HepG2 (ATCC, HB-8065)
2. Culture medium: Dulbecco's Modified Eagle Medium (Gibco, Cat. 12100-038) with 10% fetal bovine serum (Gibco, Cat. 10438-026) and 1% penicillin/streptomycin (Gibco, Cat. 15140-122)
3. Lysis buffer (Geneaid, Cat. RBD300-DG)
4. Total RNA extraction kit (Geneaid, Cat. RBD300-DG)
5. SuperScript® III Reverse Transcriptase (Invitrogen, Cat. 18080-044).
6. Primer sets (SREBP-1C, ACC, and SCD-1), and the internal control β-actin.
7. KAPA CYBR FAST qPCR Kits (2×) (KAPA Biosystems, KK4600)
8. Step One Plus (ABI)

Experiment Steps:
1. Seeding 1×10⁵ HepG2 cells with 1 mL culture medium in each well of 6-well plate.
2. Treated the HepG2 cells with 2 mg/mL or 4 mg/mL longan pericarp extract at 37° C. for 24 hours.
3. Harvesting the HepG2 cells with 300 μL the lysis buffer.
4. Collecting total RNA from the treated and the controlled HepG2 cells by the RNA extraction kit.
5. Reverse transcribed the collected RNA to the cDNA with the Reverse transcriptase.
6. Quantifying the amount of SREBP-1c, ACC and SCD-1 by real-time PCR analysis with the primer sets (table1) and the KAPA SYBR FAST qPCR Kits carried out in the ABI Step One Plus Real-Time PCR system. The PCR conditions were 95° C. for 1 seconds, 60° C. for 20 seconds (40 cycles).

TABLE 1
The primer sets

| Gene | Primer name | Number | Length (ntds) |
|---|---|---|---|
| SREBP-1c | SREBP-1c-F | SEQ ID NO: 1 | 22 |
| | SREBP-1c-R | SEQ ID NO: 2 | 21 |
| ACC | ACC-F | SEQ ID NO: 3 | 25 |
| | ACC-R | SEQ ID NO: 4 | 21 |
| SCD-1 | SCD-1-F | SEQ ID NO: 5 | 19 |
| | SCD-1-R | SEQ ID NO: 6 | 24 |
| β-actin | β-actin-F | SEQ ID NO: 7 | 21 |
| | β-actin-R | SEQ ID NO: 8 | 21 |

7. The relative quantification of gene expression is determined by the 2^{-ΔΔCt} method.
8. The statistical significance is performed by student-t-test in Excel software (*: P<0.05; **: P<0.01).

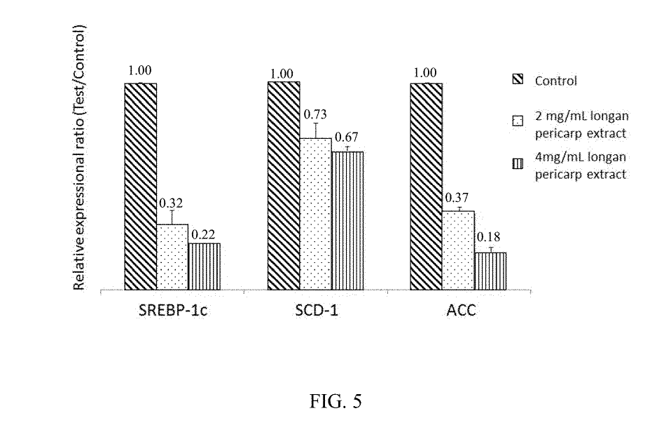

At the genetic level of fatty liver, SREBP-1c is known as a transcription factor produced when the liver cells are stimulated externally, and SREBP-1c can induce the protein production of ACC and SCD-1 which are both related to the synthesis of lipids. As shown in FIG. 5, the longan pericarp extract reduces SREBP-1c up to 78%, ACC up to 82% and SCD-1 up to 33% which is significant difference from control group. All this three proteins will increase the intrahepatic fat (triglycerides) and cause fatty liver.

In summary, nonmatter in the cell-level experiments or in the gene-level experiments, the longan pericarp extract of the present invention has the effect of regulating SREBP-1c, ACC and SCD-1, and can be used as a regulatory SREBP-1c, ACC and SCD-1 foods, drinks, medicines, reagents, or nutritional supplements, etc.

Therefore, the longan pericarp extract of the present invention can effectively inhibit the formation of fatty liver, prevent liver damage, lower the GTP value, and reduce the hepatic lipogenesis synthesis related genes to achieve the liver protection effect.

What is claimed is:

1. A method for reducing the expressions of the sterol regulatory element-binding protein 1C (SREBP-1C), acetyl-CoA carboxylase (ACC), and stearoyl-CoA desaturase 1 (SCD-1), comprising administering to a subject a composition comprising an effective amount of a longan pericarp extract, wherein the longan pericarp extract is obtained by solvent extraction of a longan pericarp, wherein the solvent is water, alcohol, or a mixture of water and alcohol.

2. The method according to claim 1, wherein the composition further comprises a pharmaceutically acceptable carrier.

3. The method according to claim 1, wherein the composition is a powder, granule, liquid, gel or paste.

4. The method according to claim 1, wherein the longan pericarp extract in the composition is at a concentration of at least 4 mg/mL.

5. The method according to claim 1, wherein a liquid-to-solid ratio of the solvent to the longan pericarp is from 20:1 to 1:1.

6. The method according to claim 1, wherein the extraction is performed from 0.5 to 3 hours.

7. The method according to claim 1, wherein the extraction is performed at a temperature from 50 to 100° C.

8. The method according to claim 1, wherein the longan pericarp extract inhibit lipogenesis.

9. The method according to claim 2, wherein the longan pericarp extract inhibit lipogenesis.

10. The method according to claim 3, wherein the longan pericarp extract inhibit lipogenesis.

11. The method according to claim 4, wherein the longan pericarp extract inhibit lipogenesis.

12. The method according to claim 5 wherein the longan pericarp extract inhibit lipogenesis.

13. The method according to claim 6, wherein the longan pericarp extract inhibit lipogenesis.

14. The method according to claim 7, wherein the longan pericarp extract inhibit lipogenesis.

15. A method for protecting liver, comprising administering to a subject a composition comprising an effective amount of the longan pericarp extract, wherein the longan pericarp extract is obtained by solvent extraction of a longan pericarp, wherein the solvent is water, alcohol, or a mixture of water and alcohol alcohols.

16. The method according to claim 15, wherein the longan pericarp extract reduce the glutamic-pyruvic transaminase (GPT) index.
